SEQUENCE LISTING (3 sequences)

SEQ ID NO: 1 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 2 - protein, 191 aa, Homo sapiens [sequence omitted]

SEQ ID NO: 3 - protein, 217 aa, Homo sapiens [sequence omitted]

(19) United States
(12) Patent Application Publication
(10) Pub. No.: US 2010/0022456 A1
(43) Pub. Date: Jan. 28, 2010

(54) Dimeric Prolactin Receptor Ligands

(75) Inventors: Leif Christensen, Roskilde (DK); Leif Norskov-Lauritsen, Tappernoje (DK); Lone Risegaard, Brondby Strand (DK); Nils Billestrup, Virum (DK); Svetlana Tarabykina, Frederiksberg (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 12/518,948

(22) PCT Filed: Dec. 20, 2007
(86) PCT No.: PCT/EP07/64329

§ 371 Date: Sep. 15, 2009

(30) Foreign Application Priority Data
Dec. 21, 2006 (EP) 06126874.4

(51) Int. Cl. A61K 38/16; C07K 14/00; A61P 35/00

(52) U.S. Cl. 514/12; 530/402; 530/409

(57) ABSTRACT

The patent application relates to antagonistic dimers of prolactin molecules and their use in treating cancer.

### FIELD OF THE INVENTION

The invention relates to novel prolactin receptor antagonist compounds, to pharmaceutical compositions comprising these compounds and to the use of the compounds for the treatment of diseases related to proliferative disorders.

### BACKGROUND OF THE INVENTION

Recent evidence suggests that prolactin may play a role as a growth promoting factor for cancer cells (Liby, et al. Breast Cancer Research and Treatment 79, 241-252 (2003); Clevenger et al. Endocrine Rev. 24,1-27 (2003)). In vitro experiments have demonstrated that the prolactin receptor antagonist G129R-hPRL shows an additive effect on the inhibition of proliferation of T47D breast cancer cells when combined with tamoxifen (Chen et al. Clin. Cancer Res. 5, 3583 (1999)). The same compound alone has shown in vivo inhibition of T47D tumour xenograft growth (Chen et al. Int. J. Oncology, 20, 813-818 (2002)).

However, high levels of these prolactin receptor antagonists are necessary to obtain effects in vivo (Goffin et al. Endocrine Rev. 26, 400-422 (2005)).

Recent attempts to generate a more potent hormone antagonist with a longer serum half-life by creating receptor antagonist multimers, surprisingly found that such receptor antagonist multimers act as receptor agonists (WO 03/089582).

Subsequent experiments (Langeheim et al. Mol Endocrinol. 20(3), 661-674 (2006) have shown that a ligand must have two functional binding sites to elicit receptor-mediated signal transduction, but this may be:

1. Site 1+Site 2 or

2. Site 1+Site 1

A demonstration of a three dimensional representation of the 1:2 ligand-receptor complex model is shown in FIG. 1.

Accordingly, joining two receptor antagonists creates a dimer with two functional binding sites capable of activating prolactin receptor mediated signal transduction.

### SUMMARY OF THE INVENTION

The present invention provides a prolactin receptor antagonist dimer comprising a first prolactin receptor binding monomer, a second prolactin receptor binding monomer and a linker, wherein each monomer comprises a first and second prolactin receptor binding site, and wherein the first monomer and the second monomer are conjugated to the linker at a position on each monomer such that the resultant dimer comprises two functional receptor binding sites.

The present invention also provides a method of treatment or prophylaxis of a proliferative disorder, which comprises administration of a prolactin receptor antagonist dimer according to the invention.

The present invention also provides the use of a prolactin receptor antagonist dimer in the manufacture of a medicament for the treatment or prophylaxis of a proliferative disorder.

The present invention also provides a pharmaceutical composition comprising a prolactin receptor antagonist dimer for use in the treatment or prophylaxis of a proliferative disorder.

### BRIEF DESCRIPTION OF THE FIGURES

FIG. 1 demonstrates a three dimensional representation of a 1:2 ligand-receptor complex model.

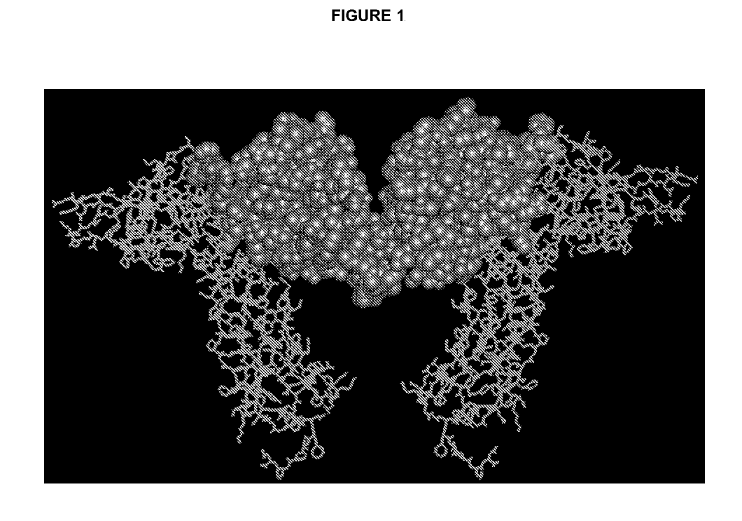

FIG. 2 demonstrates protein analysis of a variety of samples using an Agilent 2100 Bioanalyzer, Protein 230 kit. Lane 1.: PRL G129R analog (reduced), Lane 2: PRL dimer 1 (reduced), Lane 3: PRL G129R analog, Lane 4: PRL dimer 1.

FIG. 3 demonstrates the MALDI-TOF Mass/charge spectra of PRL dimer 1 using α-cyano-4-Hydroxycinnamic acid as a matrix.

FIG. 4. FIG. 4A (upper pane) demonstrates the mass/charge spectra of trypsin digested PRL dimer 1 and shows a peak corresponding to the peptide fragment (m/z=3790, z=1) encompassing disulfide linked dimer of residues 126-142 of PRL. FIG. 4B (lower pane) demonstrates the mass/charge spectra of DTT treated trypsin digested PRL dimer. 0033 is PRL 12-199 Q12S E128C.

FIG. 5 demonstrates the circular dichroism spectra of the PRL dimer.

FIG. 6 demonstrates the results of the phospho-STAT3 ELISA described in the Examples. Column 1 of the figure refers to untreated cells; Column 2: +10 nM PRL, Column 3: +10 nM PRL+10 nM PRL dimer 1; Column 4: +10 nM PRL+50 nM PRL dimer 1; Column 5: +10 nM PRL+100 nM PRL dimer 1.

FIG. 7 demonstrates the results of STAT5 tyrosine phosphorylation by Western blotting described in the Examples. Amounts of PRL and PRLR antagonist dimer are indicated. The dimer is PRL 12-199 Q12S E128C.

FIG. 8 demonstrates the results of cell migration assay described in Example 9. Column 1 of the figure represents basal migration level (no chemoattractant present) of T47D cells; Column 2: 5 nM PRL is use as chemoattractant in the lower chamber; Column 3: 5 nM PRL is used as chemoattractant in the lower chamber; 10 nM of the dimer is present in the top chamber; Column 4: 5 nM PRL is used as chemoattractant in the lower chamber; 100 nM of the dimer is present in the top chamber; Column 5: no chemoattractant added in the lower chamber; 10 nM of the dimer is present in the top chamber; Column 6: no chemoattractant added in the lower chamber; 100 nM of the dimer is present in the top chamber; Cell number in ⅙ of a 20x microscopic field±SD (n=10) is indicated.

### DETAILED DESCRIPTION OF THE INVENTION

Previous research had shown that dimers genetically engineered from monomers conjugated at the N-terminus and C-terminus acted as agonists, even if the monomers themselves were antagonists (Langeheim et al. Mol Endocrinol. 20(3), 661-674 (2006)). It was therefore presumed that a ligand with the requisite binding sites would be capable of activating receptor dimerisation and consequent signalling (WO03/089582).

The present invention provides a prolactin receptor antagonist dimer comprising a first prolactin receptor binding monomer, a second prolactin receptor binding monomer and a linker, wherein each monomer comprises a first and second prolactin receptor binding site, and wherein the first monomer and the second monomer are conjugated to the linker at a position on each monomer such that the resultant dimer comprises two functional receptor binding sites.

Prolactin is a single chain polypeptide of 199 amino acid residues with a molecular weight of about 24,000 Daltons. It is synthesised in the adenohypophysis (anterior pituitary gland), in the breast and in the decidua and has a structure similar to that of growth hormone (GH) and placental lactogen (PL). The molecule is folded due to the activity of three disulfide bonds. The sequence of human prolactin is given in SEQ ID No. 1, the sequence of human growth hormone is given in SEQ ID No. 2 and the sequence of human placental lactogen is given in SEQ ID No. 3.

A simple system is used to describe fragments and analogues of prolactin. For example, G129R-PRL designates an analogue of prolactin formally derived from prolactin by substituting the naturally occurring amino acid residue Glycine (G) in position 129 with Arginine (R). PRL(9-199) and PRL(12-199) designates a fragment formally derived from PRL by removal of the first 8 or 11 amino acids of the chain.

Human prolactin (hPRL) has two separate and different binding sites (site 1 and site 2) that each interact with a prolactin receptor to form a 1:2 ligand-receptor complex. Proper ligand-induced receptor dimerisation induces conformational changes in the receptors that bring about activation of the receptor associated Janus family of tyrosine kinases JAK2 or JAK1, which stimulate signal transducers and activators of transcription STAT5 or STAT3, respectively. Receptor activation also leads to the activation of Ras/Raf/MAPK kinase/Erk½ and phosphatidylinositol-3 kinase/Akt signalling pathways. It is primarily via these pathways that the receptors for these ligands induce cell differentiation, proliferation, and/or survival.

The binding process is reported to be sequential due to a difference in affinity between the two receptor-binding sites. Thus the higher affinity site (Site 1) interacts with the first receptor, which causes conformational changes in the ligand such that the lower affinity site (Site 2) can interact with the second receptor. This ligand-induced dimerisation of the receptors is essential for hPRL signal transduction.

Monomeric ligands with mutations that affect the structural integrity of ‘Site 2’ do not activate signal transduction because they only have one functional receptor binding site and thus, only bind to one receptor, thereby preventing proper receptor dimerisation. Such molecules do not activate the receptor and act as functional receptor antagonists.

Six prolactin receptor antagonists are currently known in the literature (Goffin et al. Endocrine Rev. 26, 400-422 (2005)):

(a) G120R/K-hGH, a variant of human growth hormone;

(b) G120R-hPL, a variant of human placental lactogen;

(c) G129R-hPRL a full-length variant of human prolactin;

(d) S179D-hPRL, a full-length variant of human prolactin;

(e) G129R-hPRL (10-199), a truncated variant of human prolactin; and

(f) G129R-hPRL (15-199), a truncated variant of human prolactin.

The term “prolactin receptor binding monomer” as used herein refers to a ligand that has the capability of binding to the prolactin receptor. The ligand may be a polypeptide, such as for instance prolactin, a prolactin analogue or another hormone or analogue with the same capability of binding to the prolactin receptor, e.g. growth hormone (GH) or a growth hormone analogue and placental lactogen (PL) or a placental lactogen analogue. In one embodiment, the prolactin receptor binding monomer is a prolactin receptor antagonist monomer.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, 3-aminomethyl benzoic acid and anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. All amino acids for which the optical isomer is not stated are to be understood to mean the L-isomer.

The term “prolactin analogue” as used herein refers to an analogue of prolactin, which analogue has the capability of binding to the prolactin receptor. In one embodiment, the prolactin analogue has an amino acid sequence having at least 80% identity to SEQ ID No. 1. In one embodiment, the prolactin analogue has an amino acid sequence having at least 85%, such at least 90%, for instance at least 95%, such as for instance at least 99% identity to SEQ ID No. 1.

The term “growth hormone analogue” as used herein refers to an analogue of growth hormone, which analogue has the capability of binding to the prolactin receptor. In one embodiment, the growth hormone analogue has an amino acid sequence having at least 80% identity to SEQ ID No. 2. In one embodiment, the growth hormone analogue has an amino acid sequence having at least 85%, such as at least 90%, for instance at least 95%, such as for instance at least 99% identity to SEQ ID No. 2.

The term “placental lactogen analogue” as used herein refers to an analogue of placental lactogen, which analogue has the capability of binding to the prolactin receptor. In one embodiment, the placental lactogen analogue has an amino acid sequence having at least 80% identity to SEQ ID No. 3. In one embodiment, the placental lactogen analogue has an amino acid sequence having at least 85%, such as at least 90%, for instance at least 95%, such as for instance at least 99% identity to SEQ ID No. 3.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percentage of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percentage sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA 89, 10915-10919 (1992) for the BLOSUM 62 comparison matrix) is also used by the algorithm.

Preferred parameters for a peptide sequence comparison include the following: Algorithm: Needleman et al., J. Mol. Biol. 48, 443-453 (1970); Comparison matrix: BLOSUM 62 from Henikoffet al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the prolactin analogue has an amino acid sequence, which sequence is at least 80% similar to SEQ ID No.1. In one embodiment, the prolactin analogue has an amino acid sequence, which sequence is at least 85%, such as at least 90%, for instance at least 95%, such as for instance at least 99% similar to SEQ ID No. 1.

In one embodiment, the growth hormone analogue has an amino acid sequence, which sequence is at least 80% similar to SEQ ID No. 2. In one embodiment, the growth hormone analogue has an amino acid sequence, which sequence is at least 85%, such as at least 90%, for instance at least 95%, such as for instance at least 99% similar to SEQ ID No. 2.

In one embodiment, the placental lactogen analogue has an amino acid sequence, which sequence is at least 80% similar to SEQ ID No.3. In one embodiment, the placental lactogen analogue has an amino acid sequence, which sequence is at least 85%, such as at least 90%, for instance at least 95%, such as for instance at least 99% similar to SEQ ID No. 3.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percentage identity and similarity would both be 50%. If, in the same example, there are 5 more positions where there are conservative substitutions, then the percentage identity remains 50%, but the percentage similarity would be 75% ((fraction ( 15/20))). Therefore, in cases where there are conservative substitutions, the degree of similarity between two polypeptides will be higher than the percentage identity between those two polypeptides.

Conservative modifications of a peptide comprising a given amino acid sequence (and the corresponding modifications to the encoding nucleic acids) will produce peptides having functional and chemical characteristics similar to those of a peptide comprising the given amino acid sequence. In contrast, substantial modifications in the functional and/or chemical characteristics of such peptide as compared to an original peptide may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol. 157,105-131 (1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

It is also understood in the art that the substitution of like amino acids may be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions”.

The term “prolactin receptor antagonist monomer” as used herein means a prolactin receptor binding monomer having antagonistic activity at the prolactin receptor, and thus acting as an inhibitor of one or more cellular processes. Such prolactin antagonistic activity may be measured as by Western blot analysis of the phosphorylation status of STAT5 as set out in Langenheim, J. F. et al, Mol Endocrinol. 20(39), 661-674 (2006).

The term “prolactin receptor antagonist dimer” as used herein means a prolactin receptor binding dimer that acts as an inhibitor of one or more cellular processes. Such prolactin antagonistic activity may be measured as by Western blot analysis of the phosphorylation status of STAT5 as set out in Langenheim, J. F. et al, Mol Endocrinol. 20(39), 661-674 (2006). The term “prolactin receptor binding dimer” as used herein means a dimer comprising two prolactin receptor binding monomers.

The term “first prolactin receptor binding site” as used herein refers to the region of prolactin with a higher affinity site that interacts with the first prolactin receptor. This region of prolactin is well known to those skilled in the art and may be interchangeably known as “Site 1” (Langenheim, J. F. et al, Mol Endocrinol. 20(39), 661-674 (2006)).

The term “second prolactin receptor binding site” as used herein refers to the region of prolactin with a lower affinity site that interacts with the second prolactin receptor. Recently it has been shown that the interaction of Site 1 with the first receptor induces conformational changes in the ligand to create a functional Site 2. This region of prolactin is well known to those skilled in the art and may be interchangeably known as “Site 2“(Langenheim, J. F. et al, Mol Endocrinol. 20(39), 661-674 (2006)).

In one embodiment, at least one of the prolactin receptor binding monomers may be truncated as compared to the parent polypeptide. The parent polypeptide should here be understood as the polypeptide from which the prolactin receptor binding monomer is derived, specifically human prolactin, human growth hormone or human placental lactogen. In one embodiment, at least one of said prolactin receptor binding monomers are PRL (10-199). In one embodiment, at least one of said prolactin receptor binding monomers are PRL (12-199). In one embodiment, at least one of said prolactin receptor binding monomers are PRL (15-199). Such truncated monomers may prevent further or alternative linker formation between cysteine residues in this region.

In one embodiment, the prolactin receptor antagonist dimer may comprise two identical prolactin receptor binding monomers. In one embodiment, the linker is positioned at the same residue position on each monomer, such that in embodiments where the dimer is a homodimer, the dimer will be symmetrical. The term “prolactin receptor antagonist homodimer” as used herein refers to a prolactin receptor antagonist dimer comprising two identical prolactin receptor binding monomers.

In one embodiment, each monomer, when part of the dimer, only has a functional first prolactin receptor binding site. For example, the linker may be positioned between residues within or adjacent to the second prolactin receptor binding site, such that after conjugation, the second binding site is disrupted

In one embodiment, the prolactin receptor binding monomer may be a prolactin receptor antagonist monomer. This may for instance be due to a disrupted second binding site as a result of mutations that affect the structural integrity of ‘Site 2’.

In one embodiment, the linker is positioned between amino acid residues 14 to 40 or amino acid residues 110 to 136 as defined by sequence alignment with SEQ ID No. 1 in at least one of the prolactin receptor binding monomers. In one embodiment, the linker is positioned between amino acid residues 14 to 40 or amino acid residues 110 to 136 as defined by sequence alignment with SEQ ID No. 1 in both prolactin receptor binding monomers.

In one embodiment, the linker is positioned at any of amino acid residues 17, 20, 21, 24, 25, 121, 125, 128, 129 and 132 as defined by sequence alignment with SEQ ID No. 1 in at least one of the prolactin receptor binding monomers. In one embodiment, the linker is positioned at any of residues 17, 20, 21, 24, 25, 121, 125, 128, 129 and 132 as defined by sequence alignment with SEQ ID No. 1 in both of the prolactin receptor binding monomers.

In one embodiment, the linker is positioned at either residue 125 or 128 as defined by sequence alignment with SEQ ID No. 1 in at least one of the prolactin receptor binding monomers. In one embodiment, the linker is positioned at either residue 125 or 128 as defined by sequence alignment with SEQ ID No. 1 in both of the prolactin receptor binding monomers.

In one embodiment, the linker is equal to or shorter than 24 bonds. By “bond” is meant a chemical bond that combines atoms and refers only to bonds in a straight chain and is not inclusive of side chain bonds or aromatic bonds.

In one embodiment, the invention provides a linker represented by the formula (I):

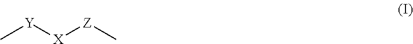

wherein
Y and Z independently represent —S—, —NH—, —CR═, —CH₂— or —CO—,

wherein R represents a hydrogen, an aryl or a C₁₋₁₀-alkyl;
X represents a linker selected from:

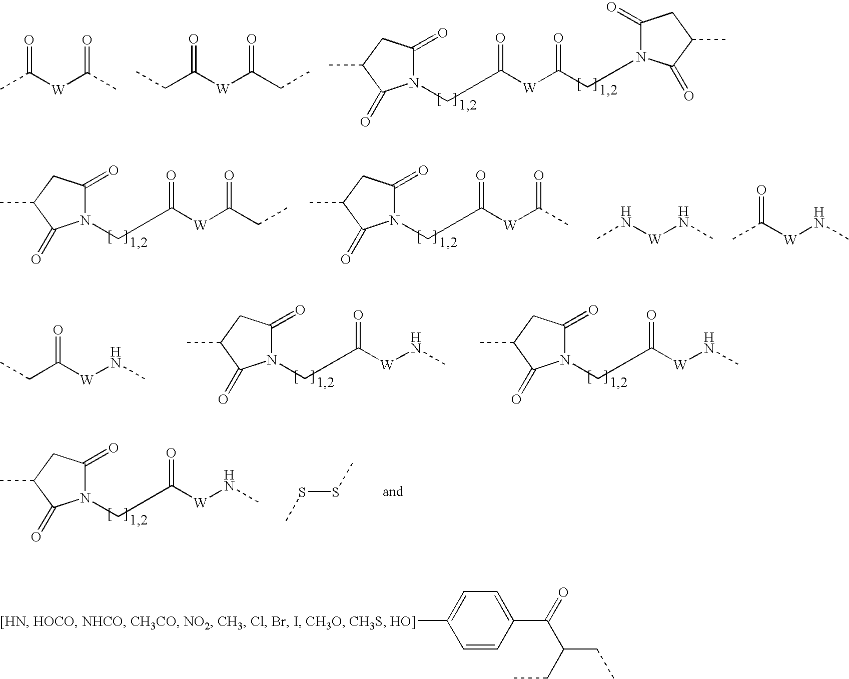

wherein
W represents; —[CH₂]ₘ—, —CH₂—[CH₂—O—CH₂]ₘ—CH₂—, —CH═CH—, —CH₂—[CH═CH]ₘ—CH₂—, —NH—CH₂—[CH₂—O—CH₂]ₘ—CH₂—NH—, —NH—CH₂—[CH₂—O—CH₂]ₘ—CH₂—,

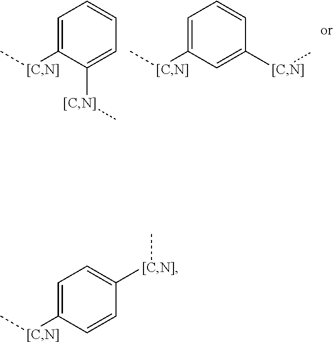

wherein m is an integer of from 1 to 22.

In the present context, the term “alkyl” is intended to indicate a straight (linear), branched or cyclic saturated monovalent hydrocarbon radical. A “C₁₋₁₀alkyl” is an alkyl having from 1 to 10 carbon atoms.

In the present context, the term “aryl” is intended to indicate a mono- or polycyclic carbocyclic aromatic ring radical with for instance 6 to 8 member atoms, or an aromatic ring system radical with for instance from 12 to 18 member atoms. Aryl is also intended to include the partially hydrogenated derivatives of the carbocyclic systems, wherein at least one ring is aromatic. Examples of such partially hydrogenated derivatives include 1,2,3,4-tetrahydro-naphthyl, fluorenyl and 1,4-dihydronaphthyl.

In one embodiment, R represents a hydrogen

In one embodiment, R represents an aryl

In one embodiment, R represents a C₁₋₁₀alkyl. In one embodiment, R represents a C₁₋₆-alkyl. C₁₋₆-alkyl groups include for instance methyl, ethyl, n-propyl, isopropyl, n-butyl, sec-butyl, isobutyl, tert-butyl, n-pentyl, 2-methylbutyl, 3-methylbutyl, 4-methylpentyl, n-hexyl, 1,1-dimethylpropyl, 1,2-dimethylpropyl, 2,2-dimethylpropyl(neopentyl) and 1,2,2-trimethylpropyl.

In one embodiment, the linker comprises an oxidative sulfide bridge formation between two cysteine residues.

In one embodiment, the linker comprises a bifunctional linker. For example, the reactive bifunctional linker precursor used for dimerization may have the formula (IA):

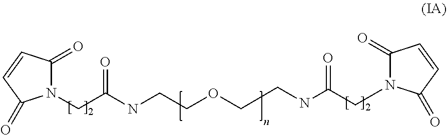

wherein n represents an integer of between 0 and 3.

A linker of formula (IA) is believed to react with free cysteine residues in the amino acid sequence of the monomer(s).

It will be appreciated that an existing residue may be substituted for a cysteine residue in the regions as defined hereinbefore in order to facilitate linker formation as required.

According to a further aspect, the invention also provides a compound of formula (II):

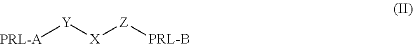

wherein
PRL-A and PRL-B each represent a radical of a polypeptide, wherein the polypeptide is capable of binding to the prolactin receptor; and
—Y—X-Z- is a linker as hereinbefore defined for compounds of formula (I).

In one embodiment, PRL-A and PRL-B independently of each other is prolactin, a prolactin analogue or another hormone or analogue with the same capability of binding to the prolactin receptor, e.g. growth hormone (GH) or a growth hormone analogue and placental lactogen (PL) or a placental lactogen analogue as described herein before In one embodiment, PRL-A and PRL-B, independently of each other, may be truncated as compared to the parent polypeptide. The parent polypeptide should here be understood as the polypeptide from which PRL-A and/or PRL-B is derived, specifically human prolactin, human growth hormone or human placental lactogen. In one embodiment, PRL-A and/or PRL-B are PRL (10-199). In one embodiment, PRL-A and/or PRL-B are PRL (12-199). In one embodiment, PRL-A and/or PRL-B are PRL (15-199).

In one embodiment PRL-A and PRL-B are identical, making the compound of formula (II) a homodimer as described herein before with regard to the prolactin receptor antagonist dimer according to the invention.

A prolactin receptor antagonist dimer according to the invention may be synthesised from prolactin receptor binding monomers via a variety of different routes using commercially available starting materials and/or starting materials prepared by conventional methods. The production of polypeptides is well known in the art. For example, polypeptides may be produced by classical peptide synthesis, e.g. solid phase peptide synthesis using t-Boc or Fmoc chemistry or other well established techniques, see e.g. Green and Wuts, “Protective Groups in Organic Synthesis”, John Wiley & Sons, 1999. The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants. The medium used to culture the cells may be any conventional medium suitable for growing the host cells, such as minimal or complex media containing appropriate supplements. The peptide produced by the cells may then be recovered from the culture medium by conventional procedures.

The DNA sequence encoding the polypeptide may suitably be of genomic or cDNA origin, for instance obtained by preparing a genomic or cDNA library and screening for DNA sequences coding for all or part of the peptide by hybridisation using specific DNA or RNA probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the polypeptide may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). The DNA sequence may also be prepared by polymerase chain reaction using specific primers, for instance as described in U.S. Pat. No. 4,683,202 or Saiki et al., Science 239, 487-491 (1988). The DNA sequence may be inserted into any vector which may conveniently be subjected to recombinant DNA procedures, and the choice of vector will often depend on the host cell into which it is to be introduced. The vector may be an expression vector in which the DNA sequence encoding the polypeptide is operably linked to additional segments required for transcription of the DNA, such as a promoter, terminator, polyadenylation signals, transcriptional enhancer sequences, and translational enhancer sequences. The vector may also comprise a selectable marker, for instance a gene the product of which complements a defect in the host cell or one which confers resistance to a drug, for instance ampicillin, kanamycin, tetracyclin, chloramphenicol, neomycin, hygromycin or methotrexate. For large scale manufacture the selectable marker may for instance be not antibiotic resistance, e.g. antibiotic resistance genes in the vector may be excised when the vector is used for large scale manufacture. Methods for eliminating antibiotic resistance genes from vectors are known in the art, see e.g. U.S. Pat. No. 6,358,705 which is incorporated herein by reference. To direct a parent peptide of the present invention into the secretory pathway of the host cells, a secretory signal sequence (also known as a leader sequence, prepro sequence or pre sequence) may be provided in the recombinant vector. The host cell into which a DNA sequence or recombinant vector is introduced may be any cell which is capable of producing the present peptide and includes bacteria, yeast, fungi and higher eukaryotic cells. The procedural steps for achieving this is all well-known to a person skilled in the art.

A variety of cellular proliferative disorders may be treated or prevented with the compounds described herein. “Proliferative disorder” refers to a disease or disorder characterised by aberrant cell proliferation, for example, where cells divide more than their counterpart normal cells. The aberrant proliferation may be caused by any mechanism of action or combination of mechanism of action. For example, the cell cycle of one or more cells may be affected such that cell(s) divide more frequently than their counterpart normal cells, or as another example, one or more cells may bypass inhibitory signals, which would normally limit their number of divisions. Proliferative disorders include, but are not limited to, carcinomas, sarcomas, leukaemias, neural cell tumours and non-invasive tumours. When used to inhibit cellular proliferation, a compound may act for instance cytotoxically to kill the cell, or cytostatically to inhibit proliferation without killing the cell.

In one embodiment, the present invention provides a method of treatment or prophylaxis of a proliferative disorder, which comprises administration of a therapeutically effective amount of a dimer according to the invention. In one embodiment, there is provided a use of the dimer according to the invention in the manufacture of a medicament for the treatment or prevention of a proliferative disorder. In one embodiment, there is provided a pharmaceutical composition comprising a dimer according to the invention for use in the treatment of a proliferative disorder.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active peptides to prevent the onset of the symptoms or complications. The patient to be treated may be a mammal, in particular a human being, but it may also include animals, such as dogs, cats, cows, sheep and pigs. It is to be understood, that therapeutic and prophylactic (preventive) regimes represent separate aspects of the present invention.

A “therapeutically effective amount” of a peptide as used herein means an amount sufficient to cure, alleviate or partially arrest the clinical manifestations of a given disease and its complications. An amount adequate to accomplish this is defined as “therapeutically effective amount”. Effective amounts for each purpose will depend on the type and severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

In one embodiment, such a proliferative disorder is a cancer.

Cancers are traditionally classified based on the tissue and cell type from which cancer cells originate. Carcinomas are considered cancers arising from epithelial cells while sarcomas are considered cancers arising from connective tissues or muscle. Other cancer types include leukaemias, which arise from haematopoietic cells, and cancer of nervous system cells, which arise from neural tissue. For non-invasive tumours, adenomas are considered benign epithelial tumours with glandular organisation while chondomas are benign tumours arising from cartilage. According to the present invention, the dimer may be used to treat proliferative disorders encompassed by carcinomas, sarcomas, leukaemias, lymphomas, neural cell tumours and non-invasive tumours.

In one embodiment, the dimer is used to treat tumours arising from variant tissue types, including, but not limited to, cancers of the bone, breast, respiratory tract (e.g. lung), brain, reproductive organs (e.g. cervix), digestive tract (e.g. gastro-intestinal tract and colorectal tract), urinary tract, bladder, eye, liver, skin, head, neck, thyroid, parathyroid, kidney, pancreas, blood, ovary, germ/prostate, neuronal tumors and metastatic forms thereof.

In one embodiment, the dimer is used to treat estrogen dependent cancer.

In one embodiment, said proliferative disorder may include proliferative disorders of the breast, which include, but are not limited to, invasive ductal carcinoma, invasive lobular carcinoma, ductal carcinoma, lobular carcinoma in situ, medular carcinoma and metastatic breast cancer.

It is to be understood that the descriptions of proliferative disorders is not limited to the conditions described above, but encompasses other disorders characterised by uncontrolled growth and malignancy. It is further understood that proliferative disorders include various metastatic forms of the tumour and cancer types described herein. The compounds of the present invention may be tested for effectiveness against the disorders described herein, and therapeutically effective regimen established. Effectiveness includes reduction or remission of the tumour, decreases in the rate of cell proliferation, induction of apoptosis, induction of cell senescence, or cytostatic or cytotoxic effect on cell growth.

The dimers described herein may be used alone, in combination with one another, or as an adjunct to, or in conjunction with, other established anti-proliferative therapies. Thus, the compounds may be used with traditional cancer therapies, such as ionisation radiation in the form of y-rays and x-rays, delivered externally or internally by implantation of radioactive compounds, and as a follow-up to surgical removal of tumours. In another aspect, the compounds may be used with other chemotherapeutic agents.

The dimers described herein may be used in combination with anti-estrogen therapies, inhibitors of growth factor receptors signalling, immunomodulators, anti-angiogenic and anti-lymphogenic therapies.

The dimers may also be administered in combination with agents useful to treat other disorders or maladies, such as steroids, membrane stabilisers and other modulators of intracellular signal transduction, protein kinase inhibitors, protein phosphotase inhibitors, cell cycle modulators and apoptosis inducing/modulating agents.

Examples of useful anti-cancer compounds are described in Merck Index, 13^{th} Ed. (O'Neil M. J. et al., ed) Merck Publish Group (2001) and Goodman and Gilmans The Pharmacological Basis of Therapeutics, 10^{th} Edition, Hardman, J. G. and Limbird, L. E. eds., pg. 1381-1287, McGraw Hill (1996).

Examples of such combination therapies may include administration of a dimer according to the present invention in combination with a medicament useful for treating cancer such as conventional chemotherapeutic agents, such as anti-metabolites (such as azathioprine, cytarabine, fludarabine phosphate, fludarabine, gemcitabine, cytarabine, cladribine, capecitabine 6-mercaptopurine, 6-thioguanine, methotrexate, 5-fluorouracil, and hydroxyurea) alkylating agents (such as melphalan, busulfan, cis-platin, carboplatin, cyclophosphamide, ifosphamide, dacarbazine, procarbazine, chlorambucil, thiotepa, lomustine, temozolamide) anti-mitotic agents (such as vinorelbine, vincristine, vinblastine, docetaxel, paclitaxel) topoisomerase inhibitors (such as doxorubicin, amsacrine, irinotecan, daunorubicin, epirubicin, mitomycin, mitoxantrone, idarubicin, teniposide, etoposide, topotecan) antibiotics (such as actinomycin and bleomycin) asparaginase, or the anthracyclines or the taxanes;

certain monoclonal antibodies (mAbs), such as Rituximab, Alemtuzumab, Trastuzumab, Gemtuzumab, Gemtuzumab-ozogamicin (Myelotarg®, Wyeth) Cetuximab (Erbitux™), Bevacizumab, HuMax-CD20, HuMax-EGFr, Zamyl and Pertuzumab and/or such as an antibody against tissue factor, killer Ig-like receptors (KIR), laminin-5, EGF-R, VEGF-R, PDGF-R, HER-2/neu, or an antibody against a tumor antigen such as PSA, PSCA, CEA, CA125, KSA, etc.;

cell cycle control/apoptosis regulators, such as compounds, which target regulators such as (i) cdc-25, (ii) cyclin-dependent kinases that overstimulate the cell cycle (for instance flavopiridol (L868275, HMR1275; Aventis), 7-hydroxystaurosporine (UCN-01, KW-2401; Kyowa Hakko Kogyo) and roscovitine (R-roscovitine, CYC202; Cyclacel)), and (iii) telomerase (such as BIBR1532 and SOT-095, as well as drugs that interfere with apoptotic pathways such as TNF-related apoptosis-inducing ligand (TRAIL)/apoptosis-2 ligand (Apo-2L), antibodies that activate TRAIL receptors, IFNα and anti-sense Bcl-2;

growth factor inhibitors, such as antibodies directed at the extracellular ligand binding domain of receptors of the epidermal growth factor receptor (EGF-R) family, and low molecular weight molecules that inhibit the tyrosine kinase domains of these receptors, for instance Herceptin, cetuximab, Tarceva and Iressa;

inhibitors of tumor vascularisation (anti-angiogenesis drugs and anti-metastatic agents) such as endostatin, angiostatin, antibodies that block factors that initiate angiogenesis (for instance anti-VEGF—Avastin), and low molecular compounds that inhibit angiogenesis by inhibiting key elements in relevant signal transduction pathways;

anti-angiogenesis drugs, such as avastin, neovastat, thalidomide, PTK787, ZK222584, ZD-6474, SU6668, PD547,632, VEGF-Trap, CEP-7055, NM-3, SU11248

hormonal agents, such as estramustine phosphate, polyestradiol phosphate, estradiol, anastrozole, exemestane, letrozole, tamoxi-fen, megestrol acetate, medroxyprogesterone acetate, octreotide, cyproterone acetate, bi-caltumide, flutamide, tritorelin, leuprorelin, buserelin or goserelin;

agents that enhance the immune response against tumor cells or virus-infected cells, such as adjuvants, for instance vaccine adjuvants such as QS21, GM-CSF and CpG oli-godeoxynucleotides, lipopolysaccharide, polyinosinic:polycytidylic acid, α-galctosylceramide or analogues thereof, histamine dihydrochloride, or aluminum hydroxide;

cytokines, such as IFN-α, IFN-β, IFN-γ, IL-2, PEG-IL-2, IL-4, IL-6, IL-7, IL-12, IL-13, IL-15, IL-18, IL-23, IL-27, IL-28a, IL-28b, IL-29, GM-CSF, Flt3 ligand or stem cell factor or an analogue or derivative of any of these;

cisplatin, tamoxifen, DTIC, carmustine, carboplatin, vinblastine, vindesine, thymosin-α, autologous LAK cells, gemcitabine;

agents that block inhibitory signalling in the immune system, such as mAbs specific for CTLA-4 (anti-CTLA-4), mAbs specific for KIR (anti-KIR), mAbs specific for LIR (anti-LIR), mAbs specific for CD94 (anti-CD94), or mAbs specific for NKG2A (anti-NKG2A);

anti-anergic agents, such as MDX-010 (Phan et al. Proc. Natl. Acad. Sci. USA 100, 8372 (2003));

antibodies against an inhibitory receptor expressed on an NK cell, a T cell or a NKT cell;

therapeutic vaccines;

agents that interfere with tumor growth, metastasis or spread of virus-infected cells; and

immunosuppressive/immunomodulatory agents such as agents with influence on T-lymphocyte homing for instance FTY-720, calcineurin inhibitors such as valspodar, PSC 833, TOR-inhibitors, sirolimus, everolimus and rapmycin.

Such combination therapy may also include administration of a dimer according to the present invention together with radiotherapy, such as external beam radiation therapy (EBRT) or internal radiotherapy (brachytherapy (BT)), typical radioactive atoms that have been used include radium, Cesium-137, Iridium-192, Americium-241, Gold-198, Cobalt-57, Copper-67, Technetium-99, Iodide-123, Iodide-131 and Indium-111

Such combination therapy may also include administration of a dimer according to the present invention together with cellular immunotherapy, which may include isolation of cells that can stimulate or exert an anti-cancer response from patients, expanding these into larger numbers, and reintroducing them into the same or another patient.

Such combination therapy may also include administration of a dimer according to the present invention together with internal vaccination, which refers to drug- or radiation-induced cell death of tumor cells that leads to elicitation of an immune response directed towards (i) said tumor cells as a whole or (ii) parts of said tumor cells including (a) secreted proteins, glycoproteins or other products, (b) membrane-associated proteins or glycoproteins or other components associated with or inserted in membranes and (c) intracellular proteins or other intracellular components.

Such combination therapy may also include administration of a dimer according to the present invention together with gene therapy, which includes transfer of genetic material into a cell to transiently or permanently alter the cellular phenotype.

The combination treatment may be carried out in any way as deemed necessary or convenient by the person skilled in the art and for the purpose of this specification, no limitations with regard to the order, amount, repetition or relative amount of the compounds to be used in combination is contemplated.

The dimers of the present invention may be generally utilised as the free substance or as a pharmaceutically acceptable salt thereof.

The term “pharmaceutically acceptable salts” refers to non-toxic salts of the prolactin receptor antagonists which are generally prepared by reacting the free base with a suitable organic or inorganic acid or by reacting the acid with a suitable organic or inorganic base.

When a dimer according to the present invention contains a free base such salts are prepared in a conventional manner by treating a solution or suspension of the dimer with a chemical equivalent of a pharmaceutically acceptable acid.

When a dimer according to the present invention contains a free acid such salts are prepared in a conventional manner by treating a solution or suspension of the dimer with a chemical equivalent of a pharmaceutically acceptable base.

Physiologically acceptable salts of a compound with a hydroxy group include the anion of said compound in combination with a suitable cation such as sodium or ammonium ion. Other salts which are not pharmaceutically acceptable may be useful in the preparation of a dimer according to the present invention and these salts are also included within the scope of the present invention.

Any salt of a dimer according to the present invention, whether a pharmaceutical acceptable salt or not, is intended to be included with the mentioning of a “dimer according to the present invention”. The invention as presented in the claims thus encompasses the dimers themselves as well as any salt thereof, for instance a pharmaceutical salt.

A dimer according to the present invention, or any of the combinations referred to above may conveniently be administered alone or in combination with pharmaceutically acceptable carriers or excipients, in either single or multiple doses, sequentially or simultaneously, by the same route of administration, or by a different route.

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995 and as it is well know in the art.

Thus, there is provided a pharmaceutical composition comprising a dimer according to the present invention, or a pharmaceutically acceptable salt, solvate, or prodrug thereof, and one or more pharmaceutically acceptable carriers, excipients, or diluents. The composition of the pharmaceutical compostion will depend on several things such as administration route, the general condition and age of the subject to be treated, the nature of the condition to be treated and the active ingredient chosen. The determination of a suitable pharmaceutical composition for a given peptide is well within the skill of a person skilled in the art.

The dimers, or compositions thereof, will generally be used in an amount effective to achieve the intended result, for example in an amount effective to treat or prevent the particular disease being treated. The compound(s) may be administered therapeutically to achieve therapeutic benefit. By therapeutic benefit is meant eradication or amelioration of the underlying disorder being treated and/or eradication or amelioration of one or more of the systems associated with the underlying disorder. Therapeutic benefit also includes halting or slowing the progression of the disease, regardless of whether improvement is realised.

The exact dosage will depend upon the frequency and mode of administration, the sex, age, weight and general condition of the subject treated, the nature and severity of the condition treated and any concomitant diseases to be treated and other factors evident to those skilled in the art. Determination of the effective dosage is well within the capabilities of those skilled in the art.

When a dimer according to the present invention (including a pharmaceutically acceptable salt, solvate or prodrug thereof) is used in combination with a second therapeutic agent active against the same disease state the dose of each compound may differ from that when the compound is used alone. Appropriate doses will be readily appreciated by those skilled in the art.

The following is a non-limiting list of embodiments of the present invention.

Embodiment 1: A prolactin receptor antagonist dimer comprising a first prolactin receptor binding monomer, a second prolactin receptor binding monomer and a linker, wherein each monomer comprises a first and second prolactin receptor binding site, and wherein the first monomer and the second monomer are conjugated to the linker at a position on each monomer such that the resultant dimer comprises two functional receptor binding sites.

Embodiment 2: A dimer according to embodiment 1, wherein the two functional binding sites are both first prolactin receptor binding sites.

Embodiment 3: A dimer according to embodiment 1 or embodiment 2, wherein at least one of said prolactin receptor binding monomers is prolactin or a prolactin analogue.

Embodiment 4: A dimer according to embodiment 3, wherein at least one of said prolactin receptor binding monomers is a prolactin analogue having at least 80% identity to SEQ ID No. 1.

Embodiment 5: A dimer according to embodiment 3, wherein at least one of said prolactin receptor binding monomers is a prolactin analogue having an amino acid sequence, which sequence is at least 80% similar to SEQ ID No. 1.

Embodiment 6: A dimer according to embodiment 1 or embodiment 2, wherein at least one of said prolactin receptor binding monomers is growth hormone or a growth hormone analogue.

Embodiment 7: A dimer according to embodiment 6, wherein at least one of said prolactin receptor binding monomers is a growth hormone analogue having at least 80% identity to SEQ ID No. 2.

Embodiment 8: A dimer according to embodiment 6, wherein at least one of said prolactin receptor binding monomers is a growth hormone analogue having an amino acid sequence, which sequence is at least 80% similar to SEQ ID No. 2.

Embodiment 9: A dimer according to embodiment 1 or embodiment 2, wherein at least one of said prolactin receptor binding monomers is placental lactogen or a placental lactogen analogue.

Embodiment 10: A dimer according to embodiment 9, wherein at least one of said prolactin receptor binding monomers is a placental lactogen analogue having at least 80% identity to SEQ ID No. 3.

Embodiment 11: A dimer according to embodiment 9, wherein at least one of said prolactin receptor binding monomers is a placental lactogen analogue having an amino acid sequence, which sequence is at least 80% similar to SEQ ID No. 3.

Embodiment 12: A dimer according to any of embodiments 3 to 11, wherein at least one of said prolactin receptor binding monomers is truncated as compared to the parent polypeptide.

Embodiment 13: A dimer according to any of embodiments 1 to 12, wherein at least one of said prolactin receptor binding monomers is a prolactin receptor antagonist monomer.

Embodiment 14: A dimer according to embodiment 13, wherein both prolactin receptor binding monomers are prolactin receptor antagonist monomers.

Embodiment 15: A dimer according to any one of embodiments 1 to 14, wherein the linker is positioned between amino acid residues 14 to 40 or amino acid residues 110 to 136 as defined by sequence alignment with SEQ ID No. 1 in at least one of the prolactin receptor binding monomers.

Embodiment 16: A dimer according to embodiment 15, wherein the linker is positioned between amino acid residues 14 to 40 or 110 to 136 as defined by sequence alignment with SEQ ID No. 1 in both prolactin receptor binding monomers.

Embodiment 17: A dimer according to any of embodiments 1 to 16, wherein the linker is positioned at any of amino acid residues 17, 20, 21, 24, 25, 121, 125, 128, 129 and 132 as defined by sequence alignment with SEQ ID No. 1 in at least one of the prolactin receptor binding monomers.

Embodiment 18: A dimer according to embodiment 17, wherein the linker is positioned at any of residues 17, 20, 21, 24, 25, 121, 125, 128, 129 and 132 as defined by sequence alignment with SEQ ID No. 1 in both of the prolactin receptor binding monomers.

Embodiment 19: A dimer according to any of embodiments 1 to 18, wherein the linker is positioned at either residue 125 or 128 as defined by sequence alignment with SEQ ID No. 1 in at least one of the prolactin receptor binding monomers.

Embodiment 20: A dimer according to embodiment 19, wherein the linker is positioned at either residue 125 or 128 as defined by sequence alignment with SEQ ID No. 1 in both of the prolactin receptor binding monomers.

Embodiment 21: A dimer according to any of embodiments 1 to 20 wherein the dimer is a homodimer.

Embodiment 22: A dimer according to embodiment 21 wherein the linker is positioned at the same residue position on each monomer.

Embodiment 23: A dimer according to any of embodiments 1 to 22, wherein said linker is equal to or shorter than 24 bonds.

Embodiment 24: A dimer according to any of embodiments 1 to 23, wherein said linker comprises an oxidative sulfide bridge formation between two cysteine residues.

Embodiment 25: A dimer according to any of embodiments 1 to 23, wherein said linker is linker represented by the formula (I):

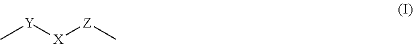

wherein
Y and Z independently represent —S—, —NH—, —CR═, —CH₂— or —CO—,

wherein R represents a hydrogen or an aryl or a linear, branched or cyclic C₁₋₁₀alkyl;
X is selected from:

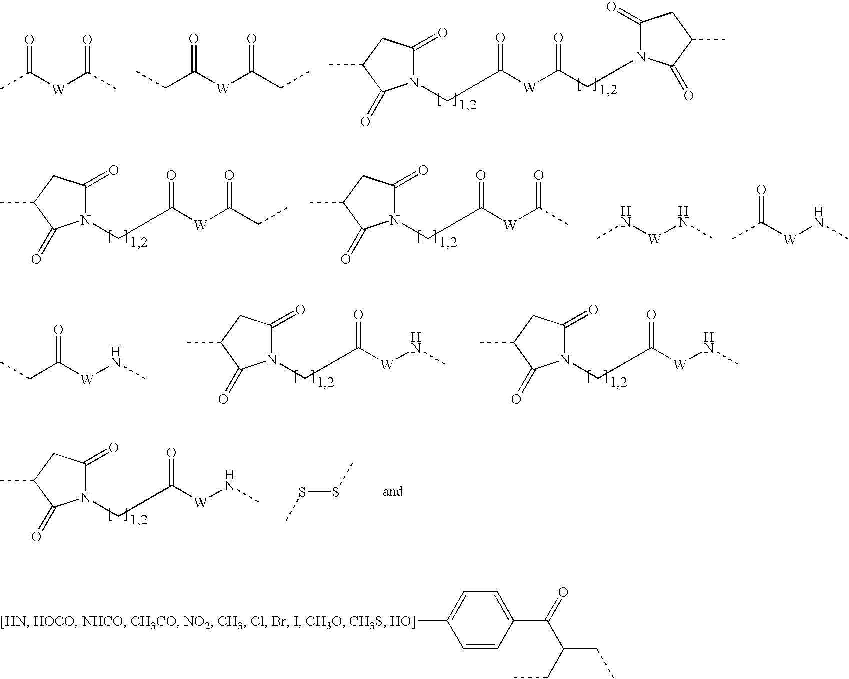

wherein
W represents; —[CH₂]ₘ—, —CH₂—[CH₂—O—CH₂]ₘ—CH₂—, —CH═CH—, —CH₂—[CH═CH]ₘ—CH₂—, —NH—CH₂—[CH₂—O—CH₂]ₘ—CH₂—NH—, —NH—CH₂—[CH₂—O—CH₂]ₘ—CH₂—,

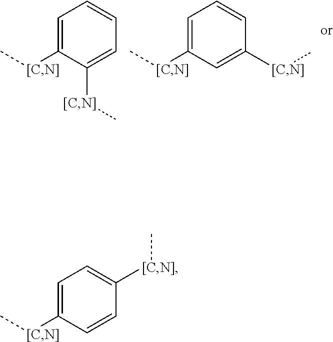

wherein m is an integer of from 1 to 22.

Embodiment 26: A dimer according to any of embodiments 1 to 25, wherein said linker is a bifunctional linker.

Embodiment 27: A dimer according to embodiment 26, wherein said linker has the formula (IA)

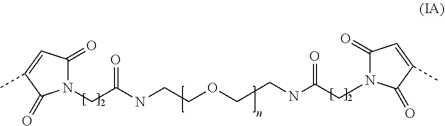

wherein n represents an integer of between 0 and 3.

Embodiment 28: A dimer according to any of embodiments 1 to 27 for use in therapy.

Embodiment 29: A dimer according to embodiment 28 for use in treating a proliferative disorder.

Embodiment 30: A dimer according to embodiment 29, wherein said proliferative disorder is a cancer.

Embodiment 31: A dimer according to embodiment 30, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

Embodiment 32: A dimer according to embodiment 31, wherein said cancer is breast cancer.

Embodiment 33: A pharmaceutical composition comprising the dimer according to any of embodiments 1 to 32.

Embodiment 34: A pharmaceutical composition according to embodiment 33 for use in the treatment or prophylaxis of a proliferative disorder.

Embodiment 35: A pharmaceutical composition according to embodiment 34, wherein said proliferative disorder is a cancer.

Embodiment 36: A pharmaceutical composition according to embodiment 35, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

Embodiment 37: Use of a dimer according to any of embodiments 1 to 27 for therapy.

Embodiment 38: Use of a dimer according to any of embodiments 1 to 27 in the treatment or prophylaxis of a proliferative disorder.

Embodiment 39: Use of a dimer according to any of embodiments 1 to 27 for the preparation of a phamaceutical composition for the treatment or prophylaxis of a proliferative disorder.

Embodiment 40: Use according to embodiment 47 or embodiment 39, wherein said proliferative disorder is a cancer.

Embodiment 41: Use according to embodiment 40, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

Embodiment 42: A method of treatment or prophylaxis of a proliferative disorder, which comprises administration of the dimer according to any of embodiments 1 to 27.

Embodiment 43: A method according to embodiment 42, wherein said proliferative disorder is a cancer.

Embodiment 44: A method according to embodiment 43, wherein said cancer is selected from an estrogen dependent cancer, breast cancer, prostate cancer, lung cancer, colorectal cancer, head and neck cancer, ovarian cancer, cervical cancer, bladder cancer, pancreatic cancer, gastrointestinal cancer, leukaemia, skin cancer, and lymphoma.

Embodiment 45: A dimer according to any of embodiments 1 to 27 for use alone or in combination with anti-estrogen therapies.

Embodiment 46: A dimer according to any of embodiments 1 to 27 for use alone or in combination with inhibitors of growth factor receptors signalling.

Embodiment 47: A dimer according to any of embodiments 1 to 27 for use alone or in combination with anti-angiogenesis therapies.

Embodiment 48: A dimer according to any of embodiments 1 to 27 for use alone or in combination with anti-lymphogenic therapies.

Embodiment 49: A dimer according to any of embodiments 1 to 27 for use alone or in combination with immunomodulating therapies.

Embodiment 50: A dimer according to any of embodiments 1 to 27 for use alone or in combination with chemotherapeutic agents.

Embodiment 51: A dimer according to and used in any of embodiments 45 to 50 for treatment of estrogen dependent cancers.

Embodiment 52: A dimer as defined and used in any of embodiments 45 to 50 for treatment of breast cancers.

Embodiment 53: A dimer as defined and used in any of embodiments 46 to 50 for treatment of prostate cancers.

Embodiment 54: A dimer as defined and used in any of embodiments 46 to 50 for treatment of lung cancers.

Embodiment 55: A dimer as defined and used in any of embodiments 46 to 50 for treatment of colorectal cancers.

Embodiment 56: A dimer as defined and used in any of embodiments 46 to 50 for treatment of head and neck cancers.

Embodiment 57: A dimer as defined and used in any of embodiments 45 to 50 for treatment of ovarian cancers.

Embodiment 58: A dimer as defined and used in any of embodiments 45 to 50 for treatment of cervical cancers.

Embodiment 59: A dimer as defined and used in any of embodiments 46 to 50 for treatment of bladder cancers.

Embodiment 60: A dimer as defined and used in any of embodiments 46 to 50 for treatment of pancreatic cancers.

Embodiment 61: A dimer as defined and used in any of embodiments 46 to 50 for treatment of gastrointestinal cancers.

Embodiment 62: A dimer as defined and used in any of embodiments 46 to 50 for treatment of leukaemia.

Embodiment 63: A dimer as defined and used in any of embodiments 46 to 50 for treatment of skin cancers.

Embodiment 64: A dimer as defined and used in any of embodiments 46 to 50 for treatment of lymphomas.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law). All headings and sub-headings are used herein for convenience only and should not be construed as limiting the invention in any way. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents. This invention includes all modifications and equivalents of the subject matter recited in the claims appended hereto as permitted by applicable law.

### Examples

The invention will be further defined by reference to the following examples, which describe the preparation of the various compounds described herein and methods for assaying their biological activity. It will be apparent to those skilled in the art that many modifications, both to the materials and methods may be practiced without departing from the scope of the invention.

### Example 1

### Protein Expression and Purification

The pET32-a(+) expression vector (Novagen, Madison Wis.) was used for expression of proteins. Recombinant Ser-hPRLR(1-210), PRL and mutated PRL monomers were produced as inclusion bodies in Escherichia coli BL21 (DE3) cells co-transfected with pACYCDuet-MetAP plasmid, which express the E. coli MetAP protein. Solubilized in 8M urea, 0.1 M Tris, 2-20 mM DTT, pH 8.5 buffer and following refolding by dilution into a 20 mM Tris, 0.05 % Tween 20, pH 8.0. Protein purification was performed using Source30Q ion exchange columns (Amersham Biosciences) followed by a macro-prep Caramic Hydroxyapatite column (BioRad) and a final size-exclusion chromatography on a Sephadex G25 column. PRL receptor was refolded in two dilution steps, first in 0.4M arginine pH 8.5 and then diluted further in 20 mM Tris, 0.05 % Tween 20, pH 8.0.

### Example 2

### Disulfide Bridged PRL 12-199 Q12S E128C Dimer

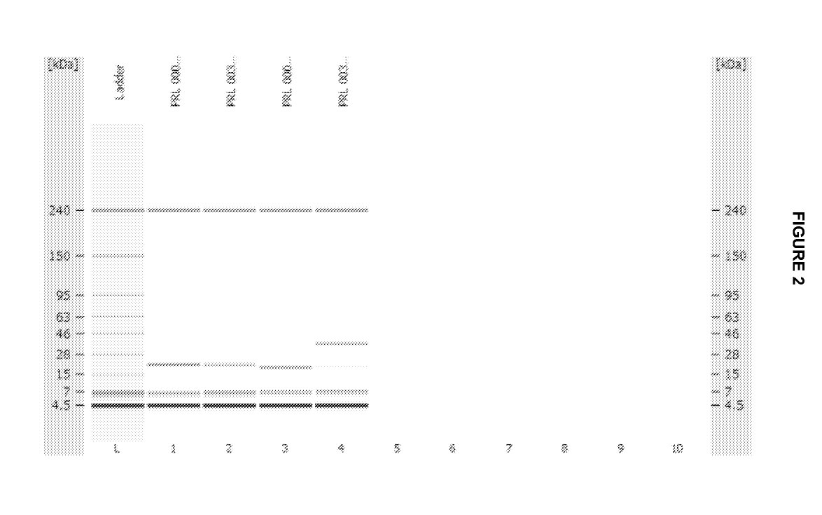

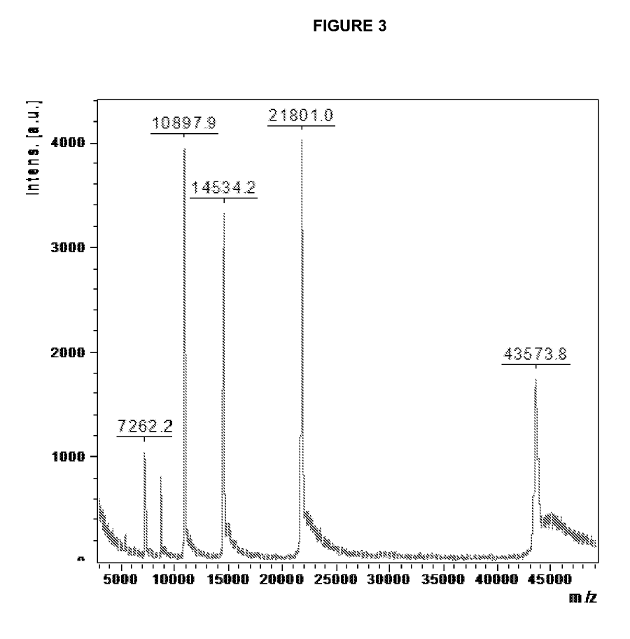

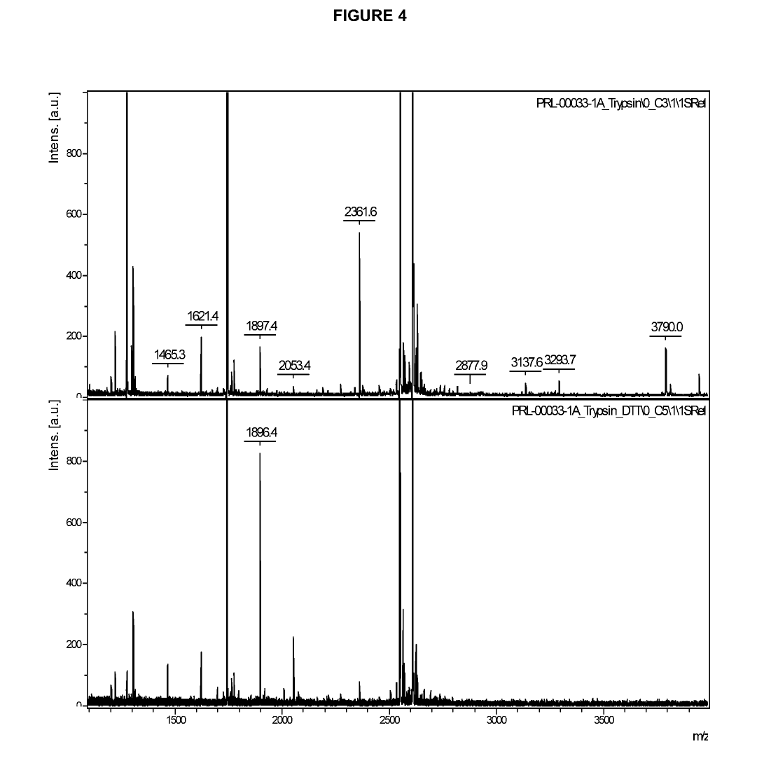

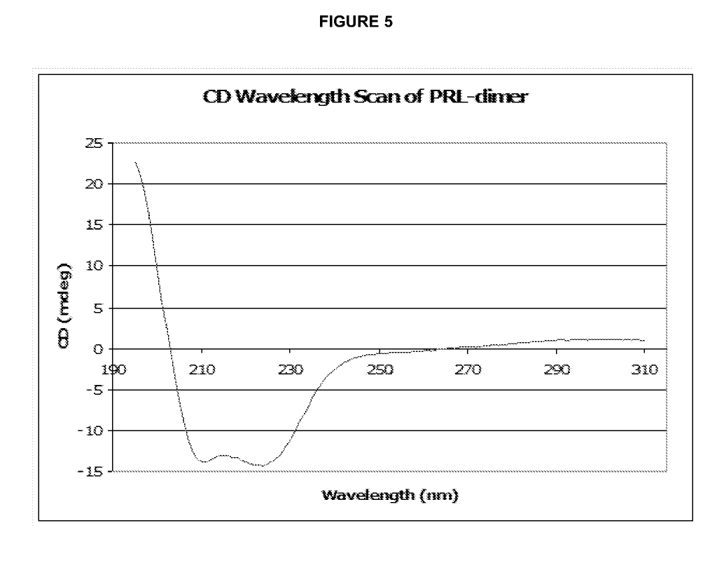

4 mg purified and freeze-dried PRL12-199 Q12S E128C monomer prepared as described in Example 1 was taken up in 3 ml 50 mM ammonium bicarbonate buffer, pH 7.8. The buffered solution was left with gentle shaking at 25° C. for 18 h in the presence of O₂ giving the desired dimeric product. The protein solution was loaded on a HiLoad 16/60 Superdex 75 prep Grade column (GE Healthcare) using 50 mM ammonium hydrogen carbonate buffer to give 1.08 mg of the desired compound. The quantification was done on a NannoDrop ND-1000 spectrophotometer, extinction coefficient (280 nm) E1% 9.04 L/gm-cm. The resultant product was characterized by SDS-electrophoresis, Agilent 2100 Bioanalyzer (FIG. 2), MALDI-TOF MS; MW (m/z, Z=1) 43573.8 (FIG. 3), Trypsin digest (FIG. 4), and Circular dichroism (FIG. 5).

### Example 3

### Disulfide Bridged PRL 12-199 Q12S R125C Dimer

5 mg purified and freeze-dried PRL12-199 Q12S E125C monomer prepared as described in Example 1 was taken up in 4 ml 50 mM ammonium bicarbonate buffer, pH 7.8. The buffered solution was left with gentle shaking at 25° C. for 18 h in the presence of O₂ giving the desired dimeric product. The protein solution was loaded on a HiLoad 16/60 Superdex 75 prep Grade column (GE Healthcare) using 50mM ammonium bicarbonate buffer to give 0.4 mg of the desired compound. The quantification was done on a NannoDrop ND-1000 spectrophotometer, extinction coefficient (280 nm) E1% 9.04 L/gm-cm. The resultant product was characterized by SDS-electrophoresis and MALDI-TOF MS; MW (m/z, Z=1) 43507.4.

### Example 4

### Disulfide Bridged PRL 12-199 Q12S S61A E128C Dimer

5 mg purified and freeze-dried PRL12-199 Q12S R125C monomer prepared as described in Example 1 was taken up in 20 ml 50 mM ammonium bicarbonate buffer, pH 7.8. The buffered solution was left with gentle shaking at 25° C. for 72 h in the presence of O₂ giving the desired dimeric product. The protein solution was loaded on a HiLoad 16/60 Superdex 75 prep Grade column (GE Healthcare) using 50 mM ammonium bicarbonate buffer to give 1.88 mg of the desired compound. The quantification was done on a NannoDrop ND-1000 spectrophotometer, extinction coefficient (280 nm) E1% 9.04 L/gm-cm. The resultant product was characterized by SDS-electrophoresis and MALDI-TOF MS; MW(m/z, Z=1) 43544.1.

### Example 5

### Bis-Maleimide dPEG3 Linked PRL 12-199 Q12S E125C Dimer

2.2 mg purified and freeze-dried PRL12-199 Q12S R125C monomer prepared as described in Example 1 was taken up in 1.2 ml 50 mM ammonium bicarbonate buffer, pH 7.8. 132 μl of a 6.37 mg/ml solution of Bis-MAL-dPEG4 (Quanta Biodesign, QBD product number 10215) in DMSO/100 mM phosphate buffer, pH:6.9 was added to a final concentration of 0.1 mg/ml of the linker. At time 1 h; 1.5 h; 2.5 h; 5 h additional 132 μl of the linker stock solution was added. The buffered solution was left with gentle shaking at 10° C. for 18 h in the presence of O₂ giving the desired dimeric product. The protein solution was purified twice using a HiLoad 16/60 Superdex 75 prep Grade column (GE Healthcare) with 50 mM ammonium bicarbonate buffer. Final yield of 60 μg of the desired compound. The quantification was done on a NannoDrop ND-1000 spectrophotometer, extinction coefficient (280 nm) E1% 9.04 L/gm-cm. The desired dimeric product was characterized and confirmed by SDS-electrophoreses and MALDI-TOF MS.

### Example 6

### Phospho-STAT3 ELISA

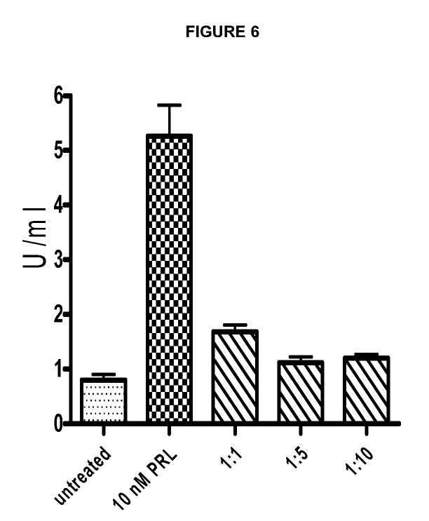

T47D cells grown to approximately 80% confluency were detached with trypsin; cell density was adjusted to 5×105/ml in full growth medium (RPMI, 10% FCS, 2 mM L-glutamin, 0.2 U/ml bovine insulin). 200 μl of this suspension was plated per well of a 96-well plate. The next day, growth medium was replaced with 150 μl starvation medium (growth medium omitting 10% FCS). The cells were starved for 24 hours prior to treatment with PRLR binding compounds. PRL and inhibitors were pre-mixed in starvation medium and 50 μl were added per well to result in 10 nM PRL and varying concentrations of PRL 12-199 Q12S S61A E128C dimer indicated at FIG. 6. The cells were incubated for 15 min at 37° C. in a humidified CO₂ incubator. Medium was removed and the cells were washed with ice-cold PBS. Lysis of cells and ELISA were performed according to BioSource STAT-3 [pY705] phospho ELISA manual.

### Example 7

### STAT5 Activation Assay

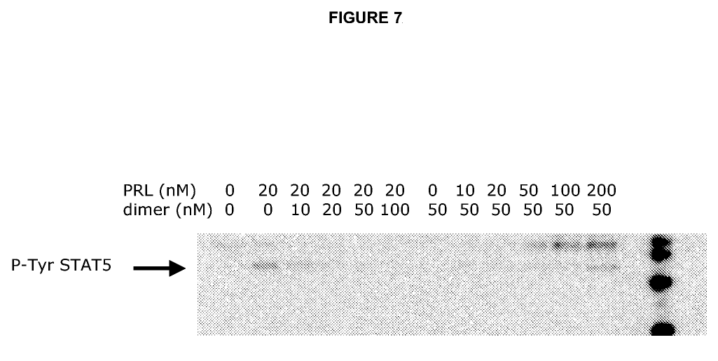

AU 565 cells were cultured for 2 days in 6-well dishes. Cells were starved for 18 hours in growth medium with <1 % FCS prior to treatment with PRLR binding compounds. The cells were incubated for 15 min at 37° C. in a humidified CO₂ incubator after addition of varying concentrations of PRL 12-199 Q12S E128C dimer as indicated in FIG. 7. Cell lysate was prepared and analyzed for STAT5 tyrosine phosphorylation by Western blotting using an anti-STAT5 [pY694] specific antibody (Cell Signalling Technologies).

### Example 8

### Prolactin Receptor Binding Assessed by Surface Plasmon Resonance Measurements (BiaCore)

The soluble form of the receptor (10 μg/ml in 10 mM sodium acetate, pH 4.0) was injected into a Biacore T100 instrument and coupled to a CM5 sensor chip by amine coupling chemistry. The immobilized level was about 500 RUs of coupled receptor. Prolactin dimers as described in Examples 2 to 5 and wild type prolactin (10, 5, 2.5, 1, 0.5, 0.1, 0.05, 0.01, 0.005, 0.001 μg in buffer; 20 mM Hepes, pH 7.4, containing 0.1 M NaCl, 2 mM CaCl₂ and 0.005% P20) were then injected over the immobilized receptor for 5 minutes at a flow rate of 30 μl/min, followed by a 10-min dissociation period during which buffer was injected, to assess receptor binding affinity. Regeneration was accomplished with 4.5 M MgCl₂ for 90 sec with a flow rate of 30 μl/min between runs. Data evaluation was performed in Biacore T100 Evaluation Software and are described in Table 1.

TABLE 1

| Compound | KD/nM |
|---|---|
| PRL(12-199) Q12S G129R | 30 |
| PRL (12-199) Q12S E128C dimer | 6 |
| PRL (12-199) Q12S R125C dimer | 20 |
| PRL (12-199) Q12S S61A E128C dimer | 3 |

### Example 9

### Cell Migration Assay

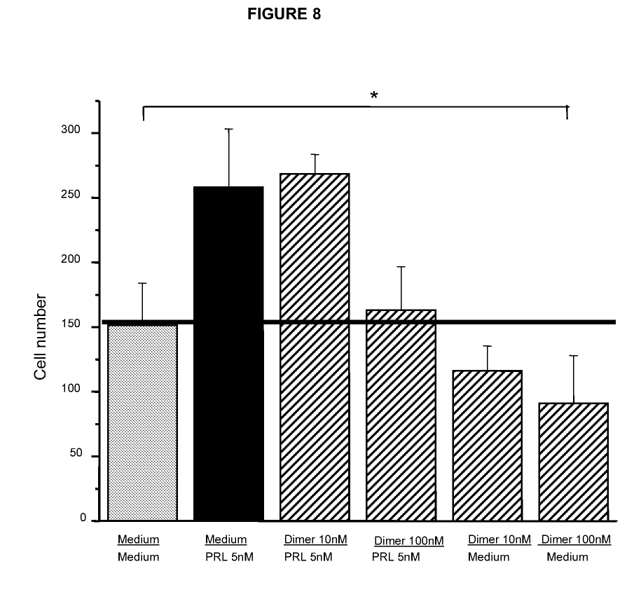

T47D cells grown to approximately 80% confluency were detached with Versen's solution. Cells were re-suspended in RPMI 1640 medium containing 0.1% BSA. Cell migration was studied in a transwell assay (modified Boyden chamber assay) through a membrane with the pore size of 12 μm (BD Biosciences). For stimulated migration 5 nM of PRL was used as chemoattractant in the lower chamber; for basal (spontaneous) migration—RPMI1640 medium containing 0.1% BSA was present in the lower chamber. PRL 12-199 Q12S S61A E128C dimer was added to the top chamber at concentration of 10 nM or 100 nM. Cell migration was analyzed for 18 hours. Cells reaching the lower chamber (attached to the membrane) were stained with hematoxilin-eozin and counted. Data represent number of cells in ⅙ of a 20× microscopic field±SD (n=10). Data is shown in FIG. 8.

1. A prolactin receptor antagonist dimer comprising a first prolactin receptor binding monomer, a second prolactin receptor binding monomer and a linker, wherein each monomer comprises a first and second prolactin receptor binding site, and wherein the first monomer and the second monomer are conjugated to the linker at a position on each monomer such that the resultant dimer comprises two functional receptor binding sites.

2. A dimer according to claim 1, wherein the two functional binding sites are both first prolactin receptor binding sites.

3. A dimer according to claim 1, wherein at least one of said prolactin receptor binding monomers is prolactin or a prolactin analogue.

4. A dimer according to claim 1, wherein the linker is positioned between amino acid residues 14 to 40 or amino acid residues 110 to 136 as defined by sequence alignment with SEQ ID No. 1 in at least one of the prolactin receptor binding monomers.

5. A dimer according to claim 4, wherein the linker is positioned between amino acid residues 14 to 40 or 110 to 136 as defined by sequence alignment with SEQ ID No. 1 in both prolactin receptor binding monomers.

6. A dimer according to claim 1, wherein the linker is positioned at any of amino acid residues 17, 20, 21, 24, 25, 121, 125, 128, 129 and 132 as defined by sequence alignment with SEQ ID No. 1 in at least one of the prolactin receptor binding monomers.

7. A dimer according to claim 6, wherein the linker is positioned at any of residues 17, 20, 21, 24, 25, 121, 125, 128, 129 and 132 as defined by sequence alignment with SEQ ID No. 1 in both of the prolactin receptor binding monomers.

8. A dimer according to claim 1, wherein said linker is equal to or shorter than 24 bonds.

9. A dimer according to claim 1, wherein said linker comprises an oxidative sulfide bridge formation between two cysteine residues.

10. A dimer according to claim 1, wherein said linker is linker represented by the formula (I): 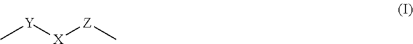
wherein
Y and Z independently represent —S—, —NH—, —CR═, —CH₂— or —CO—,
wherein R represents a hydrogen or an aryl or a linear, branched or cyclic C₁₋₁₀alkyl;
X is selected from: 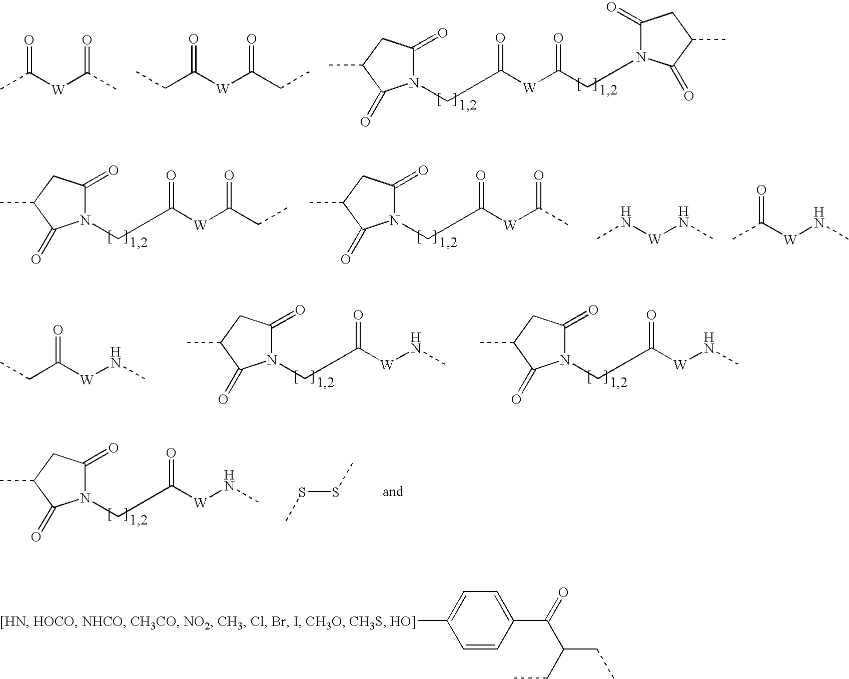
wherein
W represents; —[CH₂]ₘ—, —CH₂—[CH₂—O—CH₂]ₘ—CH₂—, —CH═CH—, —CH₂—[CH═CH]ₘ13 CH₂—, —NH—CH₂—[CH₂—O—CH₂]ₘ—CH₂—NH—, —NH—CH₂—[CH₂—O—CH₂]ₘ—CH₂—, 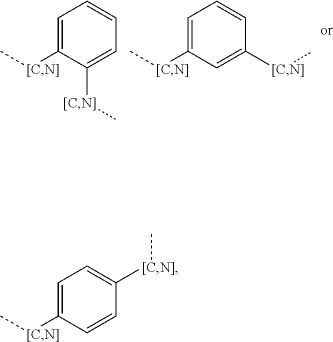
wherein m is an integer of from 1 to 22.

11. A dimer according to claim 1, wherein said linker is a bifunctional linker.

12. A dimer according to claim 11, wherein said linker has the formula (IA) 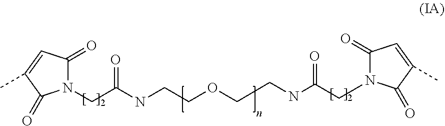
wherein n represents an integer of between 0 and 3.

13-14. (canceled)

15. A pharmaceutical composition comprising the dimer according to claim 1.

16-18. (canceled)

19. A method of treatment or prophylaxis of a proliferative disorder, which comprises administration of the dimer according to claim 1.

20. A method according to claim 19, wherein said proliferative disorder is a cancer.
